(19) United States
(12) Patent Application Publication
(10) Pub. No.: US 2019/0290714 A1
(43) Pub. Date: Sep. 26, 2019

(54) THERAPEUTIC COMPOSITIONS AND USES THEREOF FOR ALLEVIATING PAIN

(71) Applicants: Farah Kashefi, Vancouver (CA); Arash Naeini, Vancouver (CA)

(72) Inventors: Farah Kashefi, Vancouver (CA); Arash Naeini, Vancouver (CA)

(21) Appl. No.: 16/439,659

(22) Filed: Jun. 12, 2019

Related U.S. Application Data
(63) Continuation-in-part of application No. 15/239,731, filed on Aug. 17, 2016, now abandoned.

(51) Int. Cl. A61K 36/48; A61K 36/736; A61K 36/534; A61K 36/8962; A61K 36/47; A61K 36/889; A61K 9/00; A61K 47/44

(52) U.S. Cl. CPC A61K 36/48; A61K 36/736; A61K 36/534; A61K 47/44; A61K 36/47; A61K 36/889; A61K 9/0014; A61K 36/8962

(57) ABSTRACT

A therapeutic composition for skin use composed of essential oils derived from natural plant sources and at least one carrier oil. The compositions may include fenugreek essential oil, almond essential oil, mint essential oil, garlic essential oil, and castor essential oil with coconut oil as the carrier oil. The compositions are administered topically to provide pain release.

### CROSS REFERENCE TO RELATED APPLICATION

The present application is a continuation-in-part and is related to and claims priority from U.S. patent application Ser. No. 15/239,731 filed 2016 Aug. 17, which is incorporated by reference herein in its entirety and is not admitted to be prior art with respect to the present invention by the mention in this cross-reference section.

### BACKGROUND OF THE INVENTION

The following includes information that may be useful in understanding the present invention(s). It is not an admission that any of the information provided herein is prior art, or material, to the presently described or claimed inventions, or that any publication or document that is specifically or implicitly referenced is prior art.

### 1. Field of the Invention

The present invention relates generally to the field of bio-affecting and body treating compositions and more specifically relates to therapeutic compositions for skin use.

### 2. Description of Related Art

Millions of individuals suffer from acute or chronic pain every year. In 2012, a study by health economists from Johns Hopkins University estimated the total cost of chronic pain in the United States to be well over $600 billion annually. The Johns Hopkins study highlights the staggering impact of acute and chronic pain on a nation's health-care system, work productivity, and health insurance costs. Beyond the measurable economic impacts, the human suffering associated with pain is incalculable.

Conventional treatments for pain include the prescribing of pharmacologic analgesics such as acetaminophen, NSAIDs such as Ibuprofen, cyclooxygenase 2 (COX-2) inhibitors, and synthetic opioids (narcotic painkillers) such as codeine, oxycodone, and the like. It is well known that these drugs are poorly tolerated by many individuals and carry with their use substantial risks, particularly in pregnant patients, elderly patients, and individuals with certain pre-existing conditions. As examples, acetaminophen can be toxic to patients with liver disease, long-term NSAID use has been associated with gastric disorders, and narcotic painkillers can produce a myriad of serious side effects including respiratory depression, nausea, and chemical addiction. For the reasons stated above, it is clear that alternatives to conventional drug therapies, and the risks associated with their use, would be of great benefit to many who suffer from acute or chronic pain.

Several attempts have been made to solve the above-mentioned problems using alternate bio-affecting and body treating compositions, such as those found in U.S. Pat. No. 7,368,135 to Anderson disclosing “a chemical formula for the composition of a liquid topical analgesic” that “comprises at least olive oil, castor oil, grapeseed oil, almond oil, apricot kernel oil, Vitamin E, various herbal essential oils, and a stabilizer.” Other related art includes U.S. Pub. No. 20120225143 to Soliz et al.; U.S. Pub. No. 20150110903 to Khan et al.; U.S. Pub. No. 20090022826 to Shrier et al.; U.S. Pub. No. 20120141575 to Kassner et al.; U.S. Pat. No. 6,280,751 to Fletcher et al.; U.S. Pat. No. 5,032,400 to Wiersum et al.; U.S. Pat. No. 6,582,736 to Quezada; U.S. Pat. No. 6,579,543 to McClung; U.S. Pat. No. 8,623,335 to Waddington; U.S. Pat. No. 8,017,162 to Shimoda et al.; U.S. Pat. No. 6,444,238 to Weise; U.S. Pat. No. 8,765,194 to Hutsell; Canadian Pat. No. CA8017162 to Chaudhary et al.; Chinese Pat. No. CN103006852 to Shuilian; and, Chinese Pat. No. CN104258455 to Liang. This art is representative of bio-affecting and body treating compositions; however, none of the above references, taken either singly or in combination, is seen to describe the invention as claimed.

### BRIEF SUMMARY OF THE INVENTION

In view of the foregoing disadvantages inherent in the known bio-affecting and body treating composition art, the present invention provides novel therapeutic compositions for skin formulated from constituents derived from natural plant sources. Furthermore, preferred therapeutic compositions of the present system are formulated to contain no artificial colors or other synthetic chemicals. The general purpose of the present system, which will be described subsequently in greater detail, is to assist pain release.

A therapeutic treatment system is disclosed herein, in a preferred embodiment, a composition for skin is provided that preferably includes: fenugreek essential oil, almond essential oil, mint essential oil, garlic essential oil, castor essential oil, and at least one carrier oil. Moreover, in such a preferred composition, the fenugreek essential oil, the almond essential oil, the mint essential oil, the garlic essential oil, and the castor essential oil are combined in an essential-oil mixture wherein between about 55 drops and about 100 drops of such essential-oil mixture is combined with about 10 milliliters of the at least one carrier oil.

In another preferred embodiment of the present system, up to approximately 2% of the volume of the preferred composition is preferably the fenugreek essential oil, up to approximately 2% of the volume of the preferred composition is preferably the almond essential oil, up to approximately 2% of the volume of the composition is preferably the mint essential oil, up to approximately 2% of the volume of the preferred composition is preferably the garlic essential oil, and up to approximately 2% of the volume of the preferred composition is preferably the castor essential oil. Even further, the remainder of the volume of the composition is preferably the at least one carrier oil.

The present system holds significant improvements and serves as a useful therapeutic composition for topical (skin) application. Preferably, such bio-affecting and body treating compositions should assist in providing pain release, and yet would perform reliably and be manufactured at a modest expense. Thus, a need exists for new bio-affecting and body treating compositions that avoid the above-mentioned problems.

For purposes of summarizing the invention, certain aspects, advantages, and novel features of the invention have been described herein. It is to be understood that not necessarily all such advantages may be achieved in accordance with any one particular embodiment of the invention. Thus, the invention may be embodied or carried out in a manner that achieves or optimizes one advantage or group of advantages as taught herein without necessarily achieving other advantages as may be taught or suggested herein. The features of the invention which are believed to be novel are particularly pointed out and distinctly claimed in the concluding portion of the specification. These and other features, aspects, and advantages of the present invention will become better understood with reference to the following drawings and detailed description.

### BRIEF DESCRIPTION OF THE DRAWINGS

The figures which accompany the written portion of this specification illustrate embodiments and method(s) of use for the present disclosure, a therapeutic composition for skin use, formulated and operative according to the teachings of the present disclosure.

FIG. 1 is a chart illustrating preferred volumetric ratios of a group of constituents forming a therapeutic composition, according to a preferred embodiment of the present system.

FIG. 2 is a perspective view, illustrating the topical application of the therapeutic composition to a pain-affected body region of a user, according to preferred embodiments of present disclosure.

FIG. 3 is a perspective view, illustrating the topical application of the therapeutic composition to the head of a user, according to preferred embodiments of present disclosure.

FIG. 4 is a perspective view, illustrating a kit including the therapeutic composition, according to another preferred embodiment of the present system.

FIG. 5 is a flow diagram illustrating a method for providing therapeutic compositions for the skin, according to another preferred embodiment of the present system.

The various embodiments of the present invention will hereinafter be described in conjunction with the appended drawings, wherein like designations denote like elements.

### DETAILED DESCRIPTION

As discussed above, embodiments of the present disclosure relate to bio-affecting and body treating compositions and more particularly to therapeutic compositions for skin as used to relieve pain.

Generally speaking, the therapeutic compositions of the present disclosure use essential oils as an analgesic remedy to quickly ease pain and provide pain release. The disclosed compositions provide users with natural relief from muscular aches, tenseness and stiffness. A topical analgesic blended with fenugreek essential oil; almond essential oil; mint essential oil; garlic essential oil; castor essential oil; and coconut oil as career oil. May be used to give the user's body a break and ease discomfort. For the temporary relief of minor aches and pains of muscles and joints. The therapeutic compositions of the present disclosure are formulated from 100% natural ingredients with no chemical toxicants or artificial colors. Preliminary observational studies suggest that such therapeutic compositions provide muscle pain release, arthritis pain release, nerve pain release, anti-inflammatory relief (of arthrodial joint pain), and headache relief. The compositions are administered topically, generally by massaging the compositions into the skin of the affected area(s). Users have reported muscle pain release and arthritis pain release following application of the compositions.

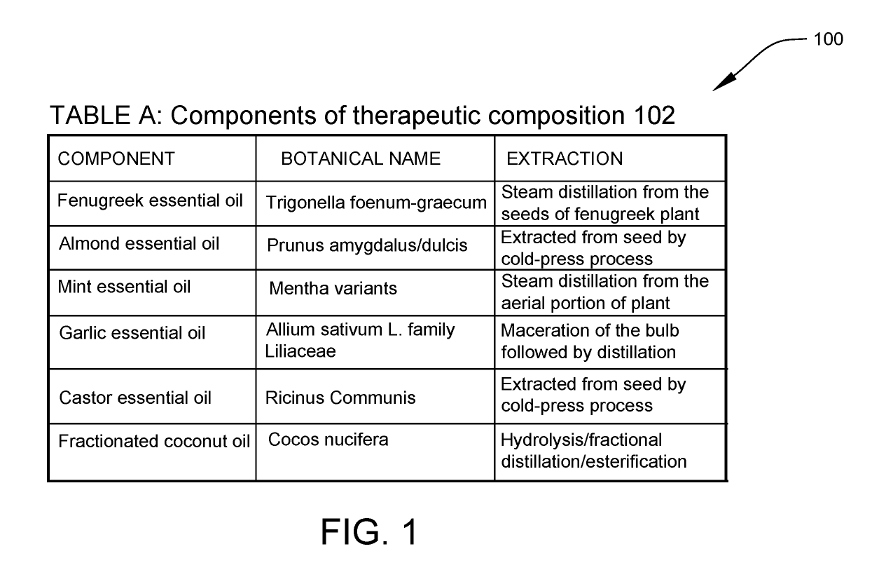

Referring now more specifically to the drawings by numerals of reference there is shown in FIGS. 1-4, various aspects of the therapeutic compositions and uses thereof for alleviating pain systems 100. In a preferred embodiment of the present system, topically-applied therapeutic compositions for the treatment of pain is provided.

In specific reference to FIG. 1, there is shown a table (TABLE A) illustrating preferred formulation of a therapeutic composition 102 according to an embodiment of the present system. TABLE A of FIG. 1 includes a list of preferred essential and carrier oils used in the formulation of therapeutic composition 102. In addition, TABLE A of FIG. 1 further includes a listing of botanical names and extraction processes suitable for use in the disclosed embodiment.

Therapeutic composition 102 is preferably formulated from combinations of fenugreek essential oil, almond essential oil, mint essential oil, garlic essential oil, castor essential oil, and at least one carrier oil. Preferred carrier oils include at least one unscented oil derived from one or more natural plant sources. In the presently-disclosed embodiment, the preferred carrier oil is fractionated coconut oil.

One of the main aspects of the present system is the preferred use of all natural (non-synthetic) essential oils in the preferred formulations, which is directed to enhancing the safety and effectiveness of therapeutic composition 102. More specifically, therapeutic composition 102 is an additive composition blend composed exclusively of natural (non-synthetic) essential oils derived from natural plant sources. The term “natural essential oils”, as used in the present disclosure, refers to concentrated volatile aromatic compounds extracted from natural plant sources. In contrast, “synthetic” essential oils are laboratory-created, often from petroleum-based chemicals and similar non-organic constituents. The use of such artificial essential oils is common in the food and cosmetic industries because of their lower cost and chemical uniformity. It is noted that essential oils suitable for use in the present embodiments of the system may be commercially sourced, so long as the volatile aromatic compounds and any neutral carrier oils used in their formulation are derived from natural plant materials and are substantially free from chemicals toxicants and artificial colors, and preservatives. The use of fractionated coconut oil as the carrier oil is preferred due in part to the exceptional quality of the oil produced by the refinement process, lack of contaminants, and apparent efficacy in the formulation of the present disclosure.

### Example Composition 1

In a first example preparation of therapeutic composition 102, between about 55 drops and about 100 drops of the following essential oils may be combined with between about 6 milliliters and about 10 milliliters of at least one carrier oil:
1) fenugreek essential oil
2) almond essential oil
3) mint essential oil
4) garlic essential oil
5) castor essential oil

In the present example, the carrier oil may be coconut essential oil.

### Example Composition 2

In a second example preparation of therapeutic composition 102, up to approximately 2% of the volume is preferably the fenugreek essential oil, up to approximately 2% of the volume is preferably the almond essential oil, up to approximately 2% of the volume is preferably the mint essential oil, up to approximately 2% of the volume is preferably the garlic essential oil, up to approximately 2% of the volume is preferably the castor essential oil, and the remainder of the volume is preferably a carrier oil in the form of fractionated coconut oil.

Coconut oil is a preferred carrier oil for use in the above-noted compositions. Coconut oil has a creamy consistency and feel when applied to skin. A preferred dilution procedure using coconut oil is as follows:
ADULTS: To dilute essential oils with the coconut oil, the five above-noted essential oils are combined in a selected ratio to form an essential-oil mixture. The therapeutic composition 102 is formed by combining between about 55 to about 100 drops of the essential-oil mixture to about 10 milliliters of coconut oil (one teaspoon is about equal to 100 drops with 100 drops equaling about 5 milliliters of coconut oil). The resulting the therapeutic composition 102 possesses a dilution rate that has been found to be both safe and effective for adult use. If the composition is being applied to sensitive areas like the underarms or face, a higher dilution of the essential oils within the coconut oil may be recommended.
If using for babies 3-24 months of age, an alternate dilution rate is recommended, which may be 1 drop of the essential-oil mixture in 4 tsp of the coconut oil.
For toddlers 2-6 years of age, an alternate dilution rate is also recommended, which may be about 1 drop of the essential-oil mixture in 2 tsp of the coconut oil.
For children 6-15 years of age, an alternate rate is also recommended, which may be about 1 drop of the essential-oil mixture in 1 tsp of the coconut oil.

The unexpected analgesic properties of the therapeutic composition 102 was discovered during experimental testing directed to the development of soothing aromatic skin preparations. Subjects using the claimed composition reported unexpected reductions in muscle pain and arthritis pain in anatomical structures underlying the areas of topical application. Similar correlations were reported by test subjects during subsequent testing.

Studies have shown that may take as little as approximately 22 seconds for the oil molecules reach the brain. In as little as two minutes, the essential oils may be detected in the blood stream. After 20 minutes the essential oils may interact with nearly every cell in the body. Although the exact mechanism of operation remains unclear, it is possible that a synergistic interaction between the complex essential oils of the claimed composition functions to enhance or magnify one or more of the analgesic properties of the constituents. Regardless of the actual mechanism, the beneficial properties of the present system appears to be unprecedented in the prior art. Those skilled in the art of the essential-oil blending will appreciate that the combination of fenugreek, almond, mint, garlic, and castor essential oils is highly unusual, which may explain the lack of prior art with regard to the claimed composition and above-described beneficial properties. Upon reading this specification, it should be appreciated that, under appropriate circumstances, considering such issues as safety, user tolerance, marketing preferences, cost, testing results, available materials, technological advances, etc., other composition arrangements such as, for example, having an increased concentration of one or more of the essential oils (up to about 18% of the volume), etc., may be sufficient.

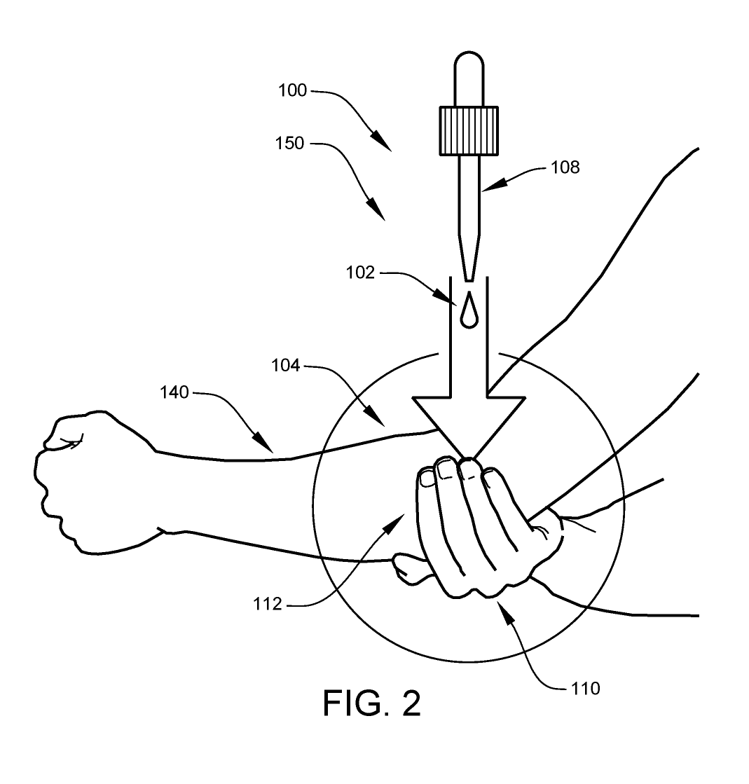

FIG. 2 is a perspective view, illustrating the topical application of therapeutic composition 102 to a pain-affected body region 104 of a user 140, according to an ‘in-use’ condition 150 of the preferred embodiment of FIG. 1. In preferred use, one or more drops of therapeutic composition 102 are applied from dispensing container 108 to the palm of the hand 110 or directly on skin 112 of the pain-affected body region 104, as shown. Therapeutic composition 102 is preferably massaged into the pain-affected body region 104 until thoroughly absorbed into skin 112. Upon reading this specification, it should be appreciated that, under appropriate circumstances, considering such issues as design preference, user preferences, marketing preferences, cost, structural requirements, available materials, technological advances, etc., other application arrangements such as, for example, utilizing topical patches infused with essential oil compositions, utilizing an aerosol applicator, utilizing a roll-on applicator, etc., may be sufficient.

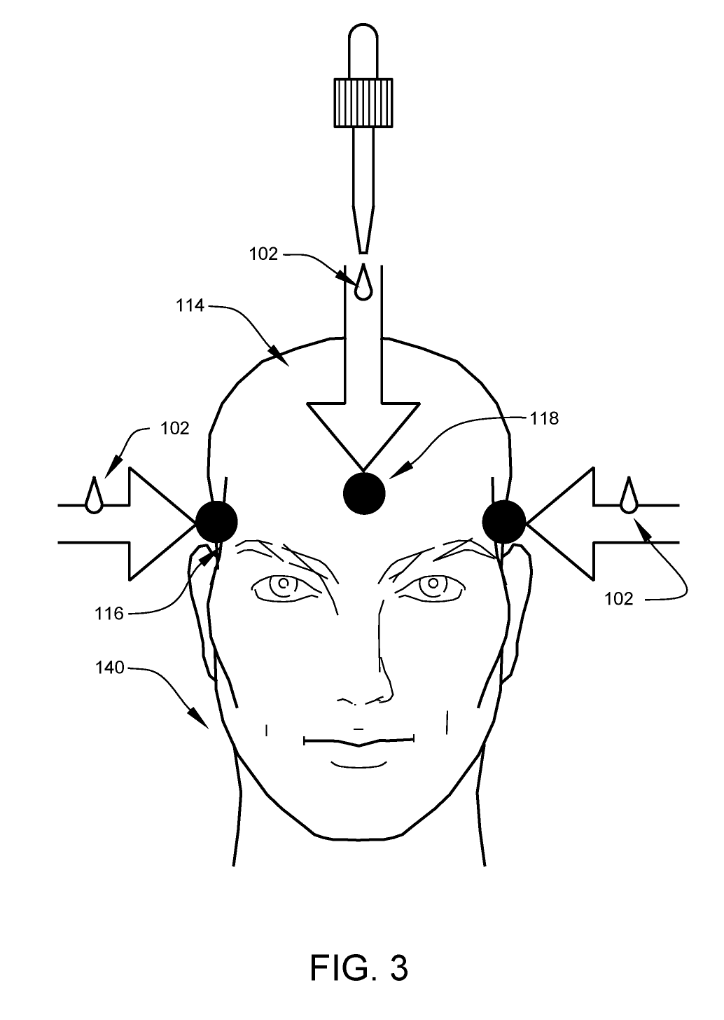

FIG. 3 is a perspective view, illustrating the topical application of therapeutic composition 102 to the head 114 of user 140, according to the preferred embodiment of FIG. 1. For headache relief, one drop of therapeutic composition 102 is applied to each temple 116 of the head 114 of user 140 and one drop of therapeutic composition 102 is applied to the top of the forehead region 118 of the head 114 of user 140, as shown. Therapeutic composition 102 is then massaged into the pain-affected body region 104 until thoroughly absorbed into the skin.

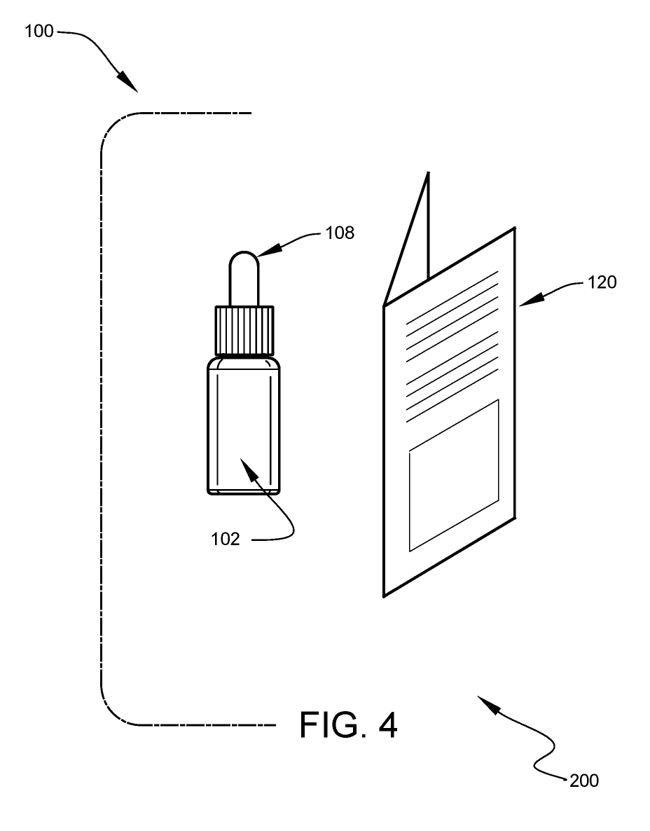

FIG. 4 is a perspective view, illustrating a kit 200 including the therapeutic composition 102, according to another preferred embodiment of the present system. Therapeutic compositions and uses thereof for alleviating pain systems 100 may be sold as a kit 200 comprising the following parts: at least one dispensing container 108 configured to hold and dispense a quantity of therapeutic composition 102 and at least one set of user instructions 120. Alternately, the kit 200 may include multiple dispensing containers 108, containing the five essential oils and carrier oil of the therapeutic composition 102. In this arrangement, the user is provided with an additional, but limited, degree of control over the concentrations of the constituent essential oils used to form the composition.

The kit 200 has instructions such that functional relationships are detailed in relation to the composition of the embodiment (such that the system can be used, maintained, or the like in a preferred manner). Information included as a part of the instructions 120 may further include the following content:

“Pain release: Give yourself some natural relief from muscular aches, tenseness and stiffness with this topical analgesic, blended with fenugreek essential oil; almond essential oil; mint essential oil; garlic essential oil; castor essential oil; and coconut oil as career oil.
To give your body a break and ease discomfort. For the temporary relief of minor aches and pains of muscles and joints.
Before applying essential oil on your skin, confirm that the essential oil(s) are properly diluted with a career oil. In this composition, coconut essential oil is used as the career oil. Before using a new essential oil, do a skin patch test to check its effects on your skin. Rub your diluted oil onto the inside of your forearm. If you don't experience any irritation or discomfort within 24 to 48 hours, the oil should be safe for you to use.
Do your research. “Not all essential oils are created equally; quality is important,
Essential oils are a nonregulated industry, meaning anyone can label an oil ‘pure’ or ‘organic.’
Make sure the oils are indigenously sourced. This means it uses ingredients from where the plant originally grows,
Verify that the label provides the plant's Latin name and common name.
   Talk to your doctor before use if you:
are pregnant
are nursing
have an existing medical condition
wish to use essential oils on children or older adults

Potential side effects of using essential oils include:
skin irritation
skin inflammation
sun sensitivity
allergic reaction
   For external use only, best apply after warm bath. Once or twice a day, focus on problematic areas.
   MASSAGE: Massaging diluted essential oil into the skin can help to loosen up muscles and alleviate pain. You can practice self-massage or opt for a professional massage using essential oils.
   Use 2 to 3 times a day as a rule, using only a couple of drops at a time.
   When used correctly, essential oils are generally safe and have powerful benefits.
   Verify that the label provides the plant's Latin name and common name.”

Therapeutic compositions and uses thereof for alleviating pain systems 100 may be supplied for sale in a wide variety of volumes, containers, and quantities for a wide assortment of applications. Upon reading this specification, it should be appreciated that, under appropriate circumstances, considering such issues as design preference, user preferences, marketing preferences, cost, structural requirements, available materials, technological advances, etc., other kit contents or arrangements such as, for example, including more or less components, customized applicators, different dispenser combinations, parts may be sold separately, etc., may be sufficient.

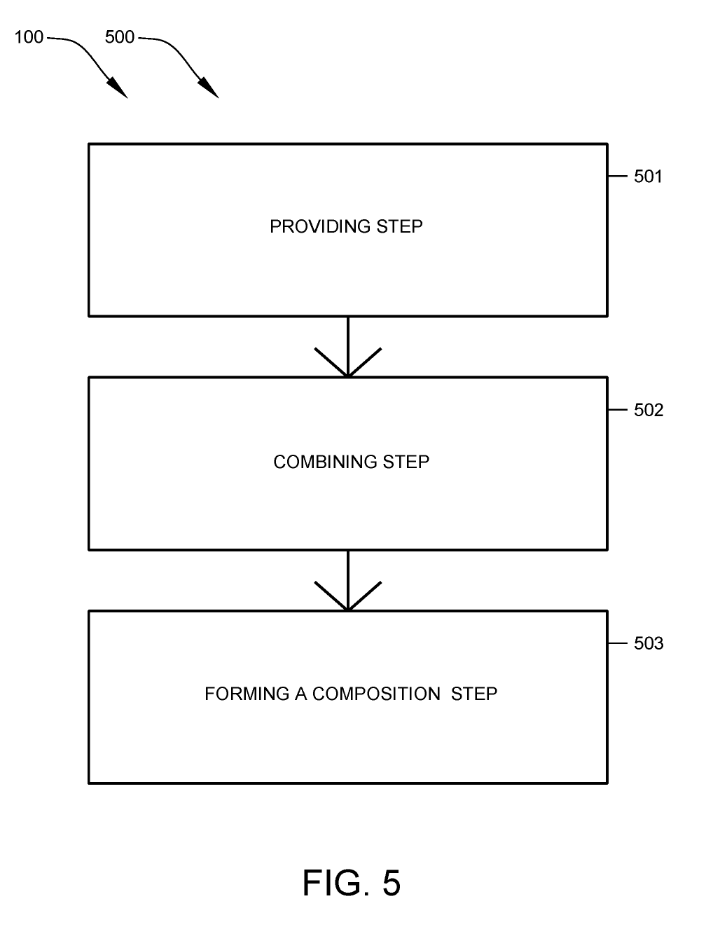

FIG. 5 is a flow diagram illustrating a method 500 for providing therapeutic compositions for the skin, according to an embodiment of the present disclosure. In particular, the method 500 may include one or more components or features of the therapeutic composition 102, as described above. As illustrated, the method 500 may include the steps of: step one 501, providing fenugreek essential oil, almond essential oil, mint essential oil, garlic essential oil, and castor essential oil; step two 502, combining the fenugreek essential oil, almond essential oil, mint essential oil, garlic essential oil, and castor essential oil in a selected combination to form an essential-oil mixture; step three 503, forming a therapeutic composition for the skin by combining between about 55 drops and about 100 drops of the essential-oil mixture with about 10 milliliters of at least one carrier oil.

It should also be noted that the steps described in the method 500 be carried out in many different orders according to user preference. The use of “step of” should not be interpreted as “step for”, in the claims herein and is not intended to invoke the provisions of 35 U.S.C. § 112(f). It should also be noted that, under appropriate circumstances, considering such issues as design preference, user preferences, marketing preferences, cost, structural requirements, available materials, technological advances, etc., other methods for providing therapeutic compositions for the skin [NOTE: e.g., different step orders within above-mentioned list, elimination or addition of certain steps, including or excluding certain maintenance steps, etc.], are taught herein.

The embodiments of the invention described herein are exemplary and numerous modifications, variations and rearrangements can be readily envisioned to achieve substantially equivalent results, all of which are intended to be embraced within the spirit and scope of the invention. Further, the purpose of the foregoing abstract is to enable the U.S. Patent and Trademark Office and the public generally, and especially the scientist, engineers and practitioners in the art who are not familiar with patent or legal terms or phraseology, to determine quickly from a cursory inspection the nature and essence of the technical disclosure of the application.

What is claimed is new and desired to be protected by Letters Patent is set forth in the appended claims:

1. A composition for skin, the composition comprising:
fenugreek essential oil;
almond essential oil;
mint essential oil;
garlic essential oil;
castor essential oil; and
at least one carrier oil.

2. The composition of claim 1, wherein the at least one carrier oil includes fractionated coconut oil.

3. The composition of claim 1, wherein:
the fenugreek essential oil, the almond essential oil, the mint essential oil, the garlic essential oil, and the castor essential oil are combined together in an essential-oil mixture; and
between about 55 drops and about 100 drops of the essential-oil mixture is combined with about 10 milliliters of the at least one carrier oil.

4. The composition of claim 3, wherein the at least one carrier oil includes fractionated coconut oil.

5. The composition of claim 1, wherein up to approximately 2% of the volume of the composition is the fenugreek essential oil.

6. The composition of claim 1, wherein up to approximately 2% of the volume of the composition is the almond essential oil.

7. The composition of claim 1, wherein up to approximately 2% of the volume of the composition is the mint essential oil.

8. The composition of claim 1, wherein up to approximately 2% of the volume of the composition is the garlic essential oil.

9. The composition of claim 1, wherein up to approximately 2% of the volume of the composition is the castor essential oil.

10. A composition for skin, the composition comprising:
fenugreek essential oil;
almond essential oil;
mint essential oil;
garlic essential oil;
castor essential oil; and
at least one carrier oil.
wherein up to about 2% of the volume of the composition is the fenugreek essential oil;
wherein up to about 2% of the volume of the composition is the almond essential oil;
wherein up to about 2% of the volume of the composition is the mint essential oil;
wherein up to about 2% of the volume of the composition is the garlic essential oil;
wherein up to about 2% of the volume of the composition is the castor essential oil; and
the at least one carrier oil includes fractionated coconut oil.

11. The composition of claim 10 further comprising a kit including:
at least one container configured to hold a quantity of the composition for skin use; and
at least one set of user instructions.

12. The composition of claim 11 wherein the kit includes a quantity of the composition.

13. The composition of claim 11 wherein the kit includes a quantity of:
the fenugreek essential oil;
the almond essential oil;
the mint essential oil;
the garlic essential oil;
the castor essential oil; and
the at least one carrier oil.

14. A method for producing a composition for skin, comprising the steps of:
providing fenugreek essential oil, almond essential oil, mint essential oil, garlic essential oil, and castor essential oil;
combining the fenugreek essential oil, almond essential oil, mint essential oil, garlic essential oil, and castor essential oil in a selected combination to form an essential-oil mixture;
forming a therapeutic composition for the skin by combining between about 55 drops and about 100 drops of the essential-oil mixture with about 10 milliliters of at least one carrier oil.
